(19) United States
(12) Patent Application Publication
(10) Pub. No.: US 2004/0019341 A1
(43) Pub. Date: Jan. 29, 2004

(54) Skin sanitary product

(76) Inventor: Joseph Chen, Yangmei Jen (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/200,318

(22) Filed: Jul. 23, 2002

(51) Int. Cl. A61F 13/15; A61F 13/20

(52) U.S. Cl. 604/379; 604/387

(57) ABSTRACT

A skin sanitary product is constructed to include an absorptive body formed of an inner water-permeable diffusion layer, an outer waterproof layer, and an intermediate absorptive layer of SAP (super absorbent polymer) mounted in between the inner water-permeable diffusion layer and the outer waterproof layer, the absorptive body having two opposite lateral sides respectively pressed together, forming a respective pommel-like stop flange, and an endless elastic band fastened to two distal ends of the absorptive body and adapted for securing the absorptive body to the body of a person.

### BACKGROUND OF THE INVENTION

1. Field of the Invention

The present invention relates skin sanitary products such as disposable diaper and disposable step-in diaper and, more specifically, to such skin sanitary products that effectively prohibit side leakage when fastened to the body of a person.

2. Description of the Related Art

A variety of skin sanitary products are commercially available for women, babies, or disabled persons to absorb waste matter discharged from the body. When the person wearing a skin sanitary product turned the body over the bed or ground, waste matter may leak out through the sides of the skin sanitary product. Further, regular disposable paints are designed for disabled persons. These disposable paints are functional, however they are expensive in comparison with disposable diapers. To a person suffering gatism, it is less comfort and not economic to use disposable paints. To some persons, it is inconvenient to fasten a disposable diaper to the body.

### SUMMARY OF THE INVENTION

The present invention has been accomplished under the circumstances in view. It is one object of the present invention to provide a skin sanitary product, which gives a wearing comfort. It is another object of the present invention to provide a skin sanitary product, which prevents side leakage. It is still another object of the present invention to provide a skin sanitary product, which greatly diminishes material consumption. According to one embodiment of the present invention, the skin sanitary product comprises an absorptive body. The absorptive body is formed of an inner water-permeable diffusion layer, an outer waterproof layer, and an intermediate absorptive layer of polymers mounted in between the inner water-permeable diffusion layer and the outer waterproof layer. The absorptive body comprises two opposite lateral sides respectively pressed together, forming a respective pommel-like stop flange for side-leakage protection. According to another embodiment of the present invention, an endless elastic band is fastened to the ends of the absorptive body, and adapted for securing the absorptive body to the user's body.

### BRIEF DESCRIPTION OF THE DRAWINGS

FIG. 1 is a schematic drawing showing the structure of an absorptive body for a skin sanitary product according to the present invention.

FIG. 2 illustrates a skin sanitary product fastened to the body of a person and covered over the private area of the person.

FIG. 3 is a cross-sectional view of the skin sanitary product shown in FIG. 2.

FIG. 4 shows the skin sanitary product made in the form of a disposable diaper according to the present invention.

FIG. 5 shows the skin sanitary product made in the form of a disposable step-in diaper according to the present invention.

### DETAILED DESCRIPTION OF THE PREFERRED EMBODIMENT

A skin sanitary product in accordance with the present invention is adapted for fastening to the body of a person to cover a part of the body and to absorb waste matter discharged from the body.

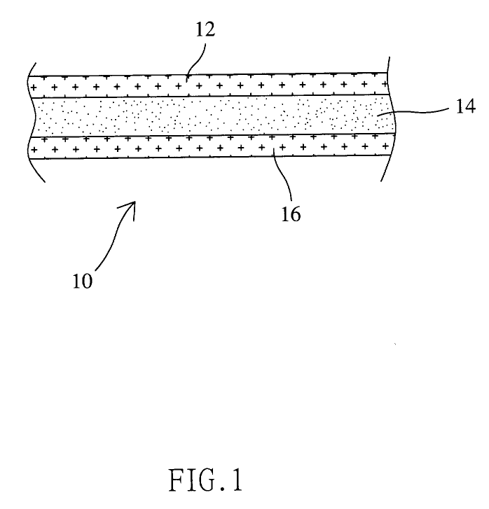

As illustrated in FIG. 1, the skin sanitary product comprises an absorptive body 10. The absorptive body 10 is comprised of an inner water-permeable diffusion layer 12, which is disposed in contact with the skin when the skin sanitary product fastened to the body of a person, an outer waterproof layer 16, and an intermediate absorptive layer of SAP (super absorbent polymer) 14 mounted in between the inner water-permeable diffusion layer 12 and the outer waterproof layer 16.

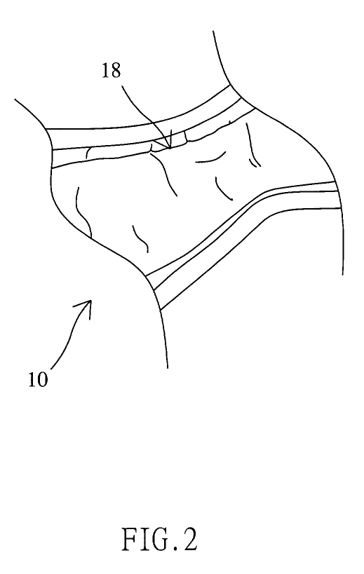

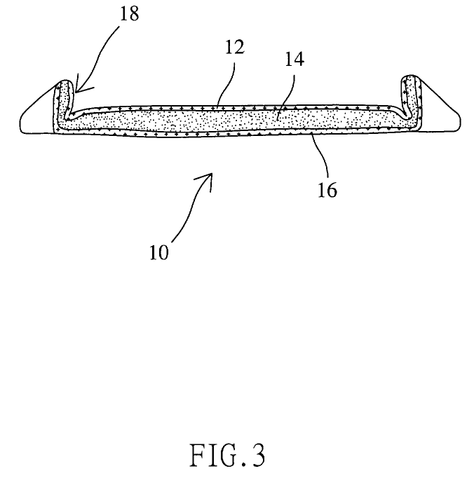

As indicated in FIGS. 2 and 3, the absorptive body 10 has two opposite lateral sides respectively pressed, forming a respective pommel-like stop flange 18 that prevents side leakage when the skin sanitary product fastened to the body of a person. According to the embodiment shown in FIG. 2, the skin sanitary product is a disposable diaper that covers the private area of the user's body, and the pommel-like stop flanges 18 of the absorptive body 10 effectively prohibit side leakage.

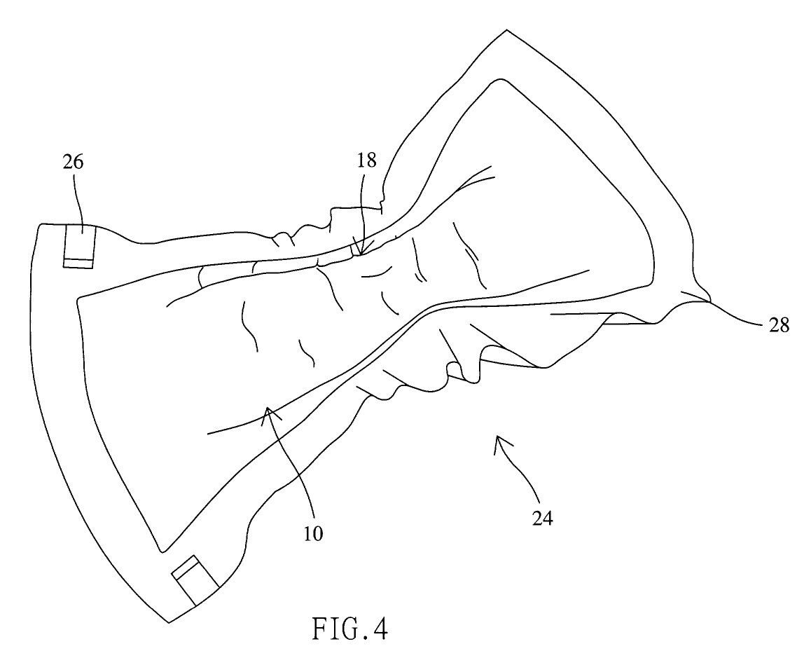

Referring to FIG. 4, the skin sanitary product is a diaper 24 having two adhesive strips 26 bilaterally disposed near one end and a front positioning portion 28. When the absorptive body 10 covered over the private part of the baby, the front positioning portion 28 is fastened to the adhesive strips 26 to secure the diaper 24 to the baby's body, keeping the pommel-like stop flanges 18 bilaterally stopped around the groin to prevent side leakage.

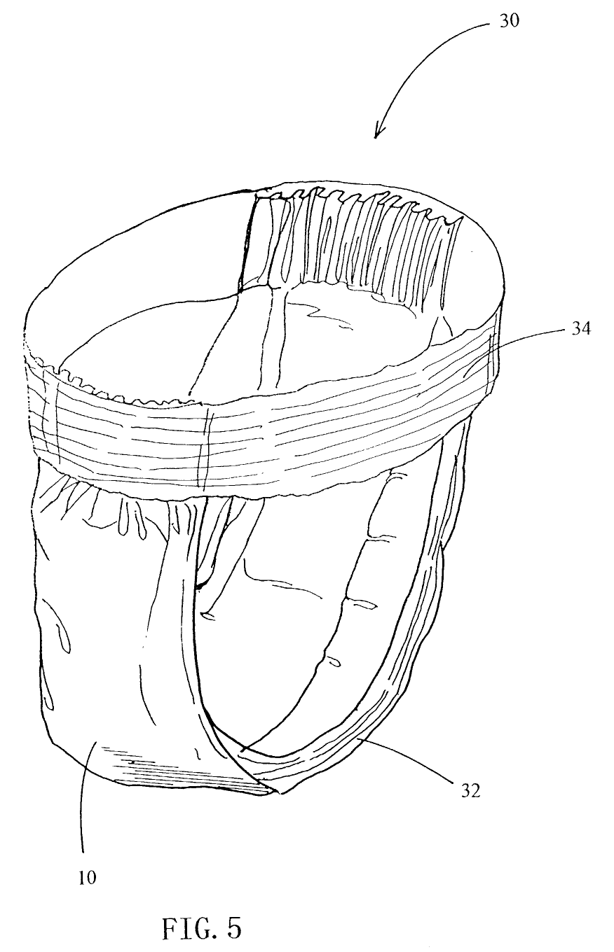

Referring to FIG. 5, the skin sanitary product is a step-in diaper 30 comprising an absorptive body 10 shaped like an elongated pad and adapted for covering the private area of the body of a person, and an endless elastic band 34 fastened to two distal ends of the absorptive body 10 and adapted for securing the absorptive body 10 to the user's body. The absorptive body 10 has two pommel-like stop flanges 32 longitudinally extended along two opposite lateral sides thereof. Further, the endless elastic band 34 is fixedly fastened to the ends of the absorptive body 10 by a heat-sealing apparatus.

As indicated above, a skin sanitary product in accordance with the present invention comprises an absorptive body having pommel-like stop flanges longitudinally disposed at two opposite lateral sides. Because the pommel-like stop flanges are formed of a part of the absorptive body by pressing, they give a wearing comfort and effectively prohibit side leakage. Further, the absorptive body can be made having elastic bands fastened to the two opposite lateral sides thereof, or made in the form of a disposable step-in diaper convenient for fastening to the user's body.

A prototype of skin sanitary product has been constructed with the features of FIGS. 1˜5. The skin sanitary diaper functions smoothly to provide all of the features discussed earlier.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A skin sanitary product comprising an absorptive body formed of an inner water-permeable diffusion layer, an outer waterproof layer, and an intermediate absorptive layer of SAP (super absorbent polymer) mounted in between said inner water-permeable diffusion layer and said outer waterproof layer, said absorptive body comprising two opposite lateral sides respectively pressed together, forming a respective pommel-like stop flange.

2. The skin sanitary product as claimed in claim 1, wherein said absorptive body comprises two adhesive tapes bilaterally disposed at one end thereof and a positioning portion disposed at an opposite end thereof and adapted for fastening to said adhesive tapes to secure said absorptive body to the body of a person.

3. The skin sanitary product as claimed in claim 1, wherein said absorptive body is a narrow elongated member adapted for covering the private part of the body of a person to absorb waste matter discharged from the body of the person, keeping the two pommel-like stop flanges thereof bilaterally stopped around the groin of the body of the person.

4. The skin sanitary product as claimed in claim 1, wherein said absorptive body has elastic bands fastened to the two opposite lateral sides thereof.

5. The skin sanitary product as claimed in claim 1 further comprising an endless band fixedly fastened to two distal ends of said absorptive body and adapted for securing said absorptive body to the body of a person.

6. The skin sanitary product as claimed in claim 5, wherein said endless band comprises an elastic band sealed to the two distal ends of said absorptive body.

7. A skin sanitary product comprising:
an absorptive body formed of an inner water-permeable diffusion layer, an outer waterproof layer, and an intermediate absorptive layer of SAP (super absorbent polymer) mounted in between said inner water-permeable diffusion layer and said outer waterproof layer, said absorptive body comprising two opposite lateral sides respectively pressed together, forming a respective pommel-like stop flange; and
an endless band fixedly fastened to two distal ends of said absorptive body and adapted for securing said absorptive body to the body of a person.

8. The skin sanitary product as claimed in claim 7, wherein said endless band comprises an elastic band sealed to the two distal ends of said absorptive body.

9. The skin sanitary product as claimed in claim 7, wherein said absorptive body is a narrow elongated member adapted for covering the private part of the body of a person to absorb waste matter discharged from the body of the person, keeping the two pommel-like stop flanges thereof bilaterally stopped around the groin of the body of the person.
